(19) United States
(12) Patent Application Publication
(10) Pub. No.: US 2007/0111953 A1
(43) Pub. Date: May 17, 2007

(54) Compositions to improve the bioavailability of polymethoxyflavones and tocotrienols for treatment of cardiovascular disease

(75) Inventors: Najla Guthrie, London (CA); Sondra Wenderoth, Winchester, VA (US)

Correspondence Address: DAVIDSON, DAVIDSON & KAPPEL, LLC, 485 SEVENTH AVENUE, 14TH FLOOR, NEW YORK, NY, 10018, US

(73) Assignee: KGK Synergize, Inc., London (CA)

(21) Appl. No.: 11/478,970

(22) Filed: Jun. 28, 2006

Related U.S. Application Data
(60) Provisional application No. 60/694,720, filed on Jun. 28, 2005.

(51) Int. Cl. A61K 31/7048; A61K 31/353; A61K 31/685; A61K 31/355

(52) U.S. Cl. 514/27; 514/78; 514/456; 514/458

(57) ABSTRACT

The present invention relates to solid compositions comprised primarily of phospholipids (also known commercially as lecithin), or enriched phospholipids, in an amount of at least 20% up to 90% by weight of the total phospholipid composition. More particularly, the present invention relates to solid phospholipid compositions that provide enhanced bioactivity of functional ingredients for the treatment, reduction and/or prevention of diseases such as hypercholesterolemia and atherosclerosis.

### BACKGROUND OF THE INVENTION

The term phospholipids usually refers to classes of amphiphilic molecules consisting of a water soluble positively charged polar group, linked to two water insoluble non-polar fatty acids by a negatively charged phosphate group. The fatty acids have between 14 and 24 carbon groups. Common types of phospholipids are phosphatidyl-choline (PC), -inositol (PI), -serine (PS) and -ethanolamine (PE). However the term phospholipid, as used herein may comprise any species of lecithin including but not limited to modified, natural, synthetic, bleached, unbleached, powdered, granular, liquid, glycero-, lyso-, polyenyl PC, PPC components, and any enriched phospholipid compounds. The term phytosterols refers to plant sterols, plant stanols and esters thereof derived from sources including but not limited to vegetable oils, and pinetree oil (known as tall oil). The term tocotrienol is meant to include alpha-, beta-, gamma-, and delta-tocotrienols. Polymethoxylated flavones are meant to include compounds derived from citrus limonoids, and citrus flavonoids.

Typical vesicle compositions known in the art consist of liposomes, nanoparticles, microspheres, etc. and serve as carriers for therapeutic and cosmetic agents. They are composed predominantly of phospholipids with exceptional tissue penetration capabilities. Such vesicles can be used as drug carriers and loaded with a large variety of molecules, such as drugs, proteins, nucleotides, etc. regardless of their solubility, charge, size or shape so long as they do not interfere with liposome formation. These different forms of vesicles are prepared using a solvent evaporation process to complex the phospholipids with the active ingredients, followed by sonication and dehydration. Such structures have been shown to improve the bioavailability of therapeutic agents. However, costs and difficulty associated with solvent recovery are not insignificant.

Thus, it is desirable to have a a solid phospholipid composition comprised primarily of phospholipids which: 1) act as a carrier for nutrients, medicaments, and/or pharmaceutical therapeutic agents, 2) which is created without the use of solvents such as for example hexane, chloroform, ether, acetone, 3) which cosolubilizes the nutrients, medicaments and/or pharmaceutical agents, 4) which forms vesicles upon hydration, and 5) which increases the bioavailability of the nutrients, medicaments and/or pharmaceutical agents useful for the treatment of hypercholesterolemia and atherosclerosis. A major advantage to a lecithin phospholipid carrier is that it is known to increase the bioavailability of other lipid ingredients. A study discussing the high bioavailability of a Coenzyme Q formulation credits the presence of non-ionic surfactants and the natural surfactant lecithin. The New Zealand Medical Journal (Oct. 8, 2004) Vol 117 No 1203.

A small set of bioflanonoids have been shown to ameliorate hypercholesterolemia and atherosclerosis by reducing the synthesis of cholesterol, LDL cholesterol and apo B protein. The present invention relates to the prevention and treatment of cardiovascular disease by the combined use of specific citrus polymethoxyflavones namely, nobiletin, tangeretin, isoscutellarein and sinensetin hereinafter referred to as PMFs. These PMFs may be combined with antioxidant tocotrienols (T-3) which are efficient free radical scavengers. Additionally, tocotrienols themselves are important factors in cholesterol reduction since they are known to inhibit the rate-limiting enzyme of the cholesterol biosynthetic pathway—coenzyme A (HMG-CoA) reductase. Thus, it is further desirable to have a solid phospholipid composition which improves bioavailability and acts as a carrier of the aforementioned PMF/T-3 combination for the reduction of cholesterol, LDL cholesterol and apo B protein.

Also, it is further desirable to have a solid composition comprised primarily of phospholipids which, in addition to the advantages given above, can be formulated into pills, tablets, powders, emulsions and/or colloids that contain nutrients, medicaments, pharmaceutical agents, and PMF/T-3 for the prevention and/or treatment of cardiovascular disease. U.S. Pat. No. 4,684,520 to Bertelli, describes the beneficial affects of an orally administered dosage of Coenzyme Q10 and phospholipids for inhibiting the formation of atherosclerotic lesions and restoring cerebral function. U.S. Pat. No. 4,780,456 to Pistolesi describes a dietetic composition for the treatment of atherosclerotic pathologies which treatment incorporates lecithin and eicosapentaenoic oils. U.S. Pat. No. 5,043,323 to Bombardelli teaches that the oral consumption of lecithin with plant flavonoids is an effective treatment for inflammation, altered platelet aggregation and other diseases. These referenced compounds may be ingested as capsules, tablets, granules, gels or syrups. However, the beneficial effects of the phospholipids are minimal in the products of these patents either because the ratio of nutritionals to phospholipid is too high, or the choice of individual fractions of phospholipids used to produce the compounds is not optimal. Therefore, it is desirable to have a solid carrier composition comprised primarily of phospholipids which can be fabricated into pills, tablets, powders, emulsions and/or colloids that contain nutrients, medicaments, pharmaceutical agents and PMF/T-3 for treatment or prevention of CVD.

In addition to its use as a carrier of nutrient and medicaments, lecithin phospholipids are known active ingredients that improve health status including, for example, reduction of cholesterol and vascular fatty deposits, while increasing the elasticity of blood vessels. Thus, it is desirable to have a nutrient carrier composition comprised primarily of phospholipids that has the added advantage of being an active ingredient.

Phospholipids of the present invention are amphiphilic compounds that spontaneously form micellular vesicles upon hydration once the critical phospholipid concentration level has been reached. Surprisingly, said vesicles are found to improve the bioavailability of lipids that have been cosolubilized with the phospholipids. Thus, it is further desirable to have a solid phospholipid carrier composition that improves the bioavailability of the nutrients, medicaments, pharmaceutical agents and PMF/T-3 for treatment or prevention of CVD.

### SUMMARY OF THE INVENTION

The present invention is directed to a composition of an amount of phospholipid equal to at least 20% by weight of the total composition and can contain an amount equal to as much as 90% by weight of the total composition. The solid phospholipid composition of the invention is a very high viscosity liquid or liquid crystal which has a continuous structure. The phospholipid composition is formed by mixing, compressing and extruding an amount of phospholipids together with nutrients, medicaments, and/or pharmaceutical agents, hereinafter called actives, under a pressure equal to at least about 100 pounds psig, at a temperature of at least 30° C., for a duration of between 10-90 seconds, with an amount of shear sufficient to cosolubilize the actives into the phospholipid composition.

The phospholipid composition thus obtained, hereinafter called a matrix, is advantageous because it cosolubilizes the actives such that the actives and the phospholipids cannot be separated except by chemical means. Matrices thus formed show improved bioavailability of the actives and can be shaped, milled, emulsified, and/or dried into dosage forms suitable for ingestion either alone or as an ingredient in a consumable solid, or liquid food product. The phospholipid matrix imparts many benefits to the consumer including, among others, acting as an antioxidant, supplying a natural source of choline, reducing platelet aggregation, improving memory retention, enhancing physical endurance, and detoxifying the liver. The solid phospholipid PMF/T-3 matrix can also contain pharmaceutical constituents which are desirable for treatment and prevention of CVD, such as statin drugs, etc.

After compression and ingestion, the PMF/T-3 matrix composition becomes hydrated by the water environment of the gut. Since the solid composition is comprised primarily of lecithin, a burst of phospholipids occurs upon hydration, meeting the critical concentration requirement to form vesicles. Thus, the present invention relates to a solid phospholipid composition which contain actives that become effectively entrapped in the phospholipid vesicles and are thus made more bioavailable.

### DETAILED DESCRIPTION OF THE PREFERRED EMBODIMENTS

The present invention relates to solid phospholipid compositions for use in the delivery of nutrients, medicaments, pharmaceutical agents, and PMF/T-3 wherein the bioavailability of said actives is increased. The compositions are made from powdered or granular phospholipids, either enhanced with phosphatides or not, having a phospholipid content with an acetone insoluble index equal to or greater than 90%. When subjected to pressure, the powdered or granular phospholipids undergo a change in state to a new form, referred to as a phospholipid matrix, which has novel and useful properties. In this solid composition, the phospholipid molecules bind to one another to form a substantially homogeneous and continuous structure similar to a meltable wax composition. The phospholipids are not, however, a meltable composition, but degrade or decompose upon heating to a temperature above nominally 70° C. The solid matrix can be used as a nutrient or as a carrier for nutrients, medicaments, and/or pharmaceutical agents.

Phospholipids have many desirable health benefits. They are an excellent source of choline, inositol, and serine which prevent accumulation of fat in the liver, control nerve impulses, aid in memory function, and integrate electrical activity across the brain zones. They are also very high in linoleic acid, an essential fatty acid. EFAs are precursors for two groups of polyunsaturated fatty acid series omega-3 and omega-6. EFAs are necessary for the normal functioning of the reproductive and endocrine systems and the breaking up of cholesterol deposits on arterial walls. Small amounts of lecithin are also used in food products as emulsifiers.

Polymethoxylated Flavones

Flavonoids are a group of low molecular weight polyphenolic compounds with a broad range of biological activities. They are naturally found in the heavily pigmented yellow, red, and purple fruits, vegetables, teas, and wines as well as nuts, and seeds. Many of the nutritional actions of foods are directly related to their flavonoid content. In addition to acting as free-radical scavengers, they may exhibit anti-inflammatory properties or prevent/slow the development of some cancers. For purposes of the present invention examples of naturally occurring polymethoxyflavones include, but are not limited to, nobiletin, tangeretin, 5-desmethylnobiletin, tetramethylscutellarein, and sinensetin.

The mixture and use of at least one limocitrin derivative, one polymethoxyflavone, and one tocotrienol is known for the treatment of cardiovascular diseases. U.S. Pat. No. 6,251,400 to Guthrie N, discusses the ability of citrus flavonoids to lower LDL cholesterol, inhibit liver cholesterol and apo-B synthesis. Kurowska EM, et al has shown that polymethoxylated flavones are novel flavonoids with cholesterol- and triacylglycerol-lowering potential. J Agric Food Chem. (2004) 52, 2879-86. Whitman S C et al. showed that nobiletin, a citrus flavonoid isolated from tangerines, inhibited (50-72%) acetylated LDL metabolism. These findings suggest that in addition to reducing plasma cholesterol concentrations, the polymethoxyflavone nobiletin may prevent atherosclerosis at the level of the vascular wall by inhibiting macrophage foam-cell formation. Research performed by Lee C H, suggest that the anti-atherogenic effect of citrus flavonoids, naringin and naringenin, is involved with a decreased hepatic ACAT activity. Biochem Biophys Res Commun. (2001) 284, 681-688. A solid phospholipid matrix delivery system to improve the bioavailability of actives such as polymethoxyflavones for cholesterol reduction and atherosclerotic disease has not been reported.

Tocotrienols

Tocotrienols are isomers of vitamin E that are known to reduce serum cholesterol, prevent atherosclerosis and improve vascular blood flow. Theriault A, et al report that tocotrienol has been shown to possess novel hypocholesterolemic effects together with an ability to reduce the atherogenic apolipoprotein B and lipoprotein(a) plasma levels (1999) Clin Biochem 32:309-19. More recently, he suggested that alpha-T3 is a potent and effective agent in the reduction of cellular adhesion molecule expression and monocytic cell adherence. Atherosclerosis. (2002), 160(1):21-30. Parker R A, et al reported that tocotrienols act by suppressing the activity of HGM-CoA reductase. J Biol Chem (1993), 268:11230-8. Also, Pearce B C, et al J Med Chem (1992) 35:3595-606. U.S. Pat. No. 4,603,142 to Burger W discloses the use of alpha tocotrienol to reduce cholesterol. Lievense in WO 96/38047, discloses a fat based food containing tocotrienol, a phytosterol and/or oryzanol to lower blood cholesterol. U.S. Pat. No. 6,864,280 to Igarashi O, teaches the use of gamma tocotrienol for treating hypertension and cardiac disease. Tan D, found that palm-oil-vitamin E concentrate had a positive effect on the serum and lipoprotein lipids in humans, Am. J. Clin. Nutr (1991) 53:1027S-1030S. Minhajuddin M, reports that tocotrienol modulates atherogenic lipid profiles and antioxidants in hypercholesterolemia. Food Chem Toxicol. (2005) 43, 747-53. A solid phospholipid matrix delivery system to improve the bioavailability of tocotrienols alone or in combination with polymethoxyflavones for cholesterol reduction and atherosclerotic disease has not been reported.

Plant Sterols

Phytosterols are known to decrease blood cholesterol and the risk of cardiovascular disease. These compounds are of plant origin but have a chemical structure very much like cholesterol which occurs in animal tissue. Upon ingestion, only about 10% of the sterols that arrive at the absorption sites of the small intestine are actually assimilated. The remaining 90% are not absorbed, but remain at the sites for long periods of time acting as a barrier to the absorption of cholesterol. According to the FDA, the minimum dose of phytosterols is 400 mg and the intake of sterols must be at least 800 mg to reduce the risk of coronary heart disease. Phytosterols are hydrophobic, and must be solubilized in a fat to be effective. Thus, plant sterols, stanols, and their esters are typically delivered in margarines, salad dressings, or other fatty foods. These cholesterol lowering foods usually contain fatty acids of stanols since they are more readily solubilize with dietary fats. Health professionals and consumer groups have shown resistance to a cholesterol reducing regimen that includes a requirement to consume fat. In order to improve this image, research was conducted using no fat substrates. Jones et al showed that a dietary intake of phytosterols in a low-fat beverage format is not efficacious for lipid lowering modification. J Lipid Res (2003), 44, 1713-1719. The present invention utilizes hypocholesterolemic phospholipids rather than triglycerides as the lipid carrier of free phytosterols.

U.S. Pat. Nos. 6,063,776 and 5,932,562 to Ostlund describe a formulation containing plant stanols to reduce the absorption of dietary cholesterol. The process is one of forming liposomes. WO Patent #9956729 to Sjoeberg, K describe a cholesterol lowering composition of sterols which are delivered in a high molecular material. WO Patent #9953925 to Stohler, E, describes a cholesterol lowering combination of phytosterols in a micellular phase using lecithin and creating liposomes. U.S. Pat. No. 6,136,349 to Karppanen teaches the incorporation of phytosterols and certain minerals into foods to lower serum cholesterol. Ostlund, R E et al, reported that sitostanol reduced cholesterol absorption at doses lower than reported previously, but only if presented in lecithin micelles. Am J of Clinical Nutrition (1999) 70, 826-831. A solid phospholipid matrix delivery system to improve the bioavailability of polymethoxyflavones, tocotrienols and phytosterols for the reduction of cholesterol and atherosclerotic disease has not been reported.

Lecithin

It has been known to use lecithin phospholipids as ingredients to reduce serum cholesterol in animals. U.S. Pat. No. 6,773,719 to Rodrigueza provides for the reverse transport of cholesterol from peripheral tissues to the liver by administering empty phospholipid liposomes for the treatment of dislipidemia. Wilson, T. et al found that when administered in conjunction with the American Heart Association Step I diet, lecithin enhances the reduction of plasma cholesterol and LDL in monkeys, and that phospholipids are effective in reducing total cholesterol and triglycerides in hypercholesterolemic hamsters. Atherosclerosis (1998) 140: 147-153). The positive effects of lecithin phospholipids in reducing arterial plaque, besides decreasing LDL cholesterol, and increasing HDL cholesterol, were shown by Wojcicki, J. et al, Phytotherapy Research (1995) 9, 597-599. The positive effects on rabbits of a lecithin and cholesterol diet was reported by Hunt, C et al, British J of Experimental Pathology (1985) 66, 35-46. FASEB reported on the effects of choline and lecithin on neurological and cardiovascular system. (August 1981). O'Brien, B et al, found that soy phospholipids reduced LDL cholesterol in humans. Lipids (1993) 28, 7-12. Polichetti, E et al report that soy lecithin phospholipids stimulate the apo AI-HDL system. J of Nutritional Biochemistry (1998) November, 659-664. Iwata, T et al, found that soy or safflower phospholipids markedly inhibited the absorption of dietary cholesterol in the intestines of rats. J Nutr. Sci. Vitaminol. (1993) 39, 63-71. Simons, L A et al, present evidence that lecithin reduces serum cholesterol by acting as a source of linoleic acid. Aust. N. Z. J of Medicine (1972), 7, 262-266.

Thus, it is advantageous that the phospholipid carrier matrix of the present invention is itself a hypolipidemic agent. Also, lecithin is known to be synergistic with vitamin E (tocotrienol is an isomer) which is an effective antioxidant used to neutralize oxidized LDL lipids that damage the lining of the arterial walls, Sugino, H et al, J Agric Food Chem. (1997) 45, 551-554.

Thus, a solid phospholipid matrix delivery system to improve the bioavailability of the actives in PMF/T-3 containing >15% tangeretin, >15% nobiletin, >1% sinensetin, >0.5% tetramethylscutellarein, >2% desmethylinobiletin, >14% tocotrienols for cholesterol reduction and atherosclerotic disease has not been reported.

All references listed herein are hereby incorporated by reference in their entireties for all purposes. Also, U.S. Ser. No. 11/135,693, filed May 24, 2005, U.S. Ser. No. 11/135,675, filed May 24, 2005 and U.S. Ser. No. 11/135,694, filed May 24, 2005, are hereby incorporated by reference in their entireties for all purposes.

### EXAMPLES

The following examples illustrate the use of the invention for the prevention and/or treatment of cardiovascular diseases and disorders. It will be appreciated by one skilled in the art that the invention is not limited to the following examples.

### Example 1

Eighty male Sprague-Dawley rats weighing between 310 and 340 grams were supplied by Harlan Tecklad. At the initiation of the study, the rats were fed a purified low alpha tocopherol diet for 2 weeks as a baseline. Natural source a-tocopherol (Covitol F1300) was supplied by Henkel Corp., and was assayed at 1314 IU/gr which is equivalent to 882 mg/gr. The four diets consisted of: Control: Low α-tocopherol diet (11.9 mg/kg of feed); Group 2: Normal α-tocopherol diet (71 mg/kg); Group 3: Phospholipid+Normal α-tocopherol (71 mg/kg) Matrix; Group 4: Phospholipid+Polysorbate+Normal α-tocopherol (71 mg/kg) Matrix.

Blood samples were collected at weeks 0, 2 after a 12 hour fast, and the red blood cells were separated at low speed centrifugation. The data were analyzed by one way ANOVA and treatment differences identified by Tukeys. The baseline data between the groups showed no significant differences in plasma concentrations. The plasma α-tocopherol concentrations were significantly (P<0.05) greater at 2 weeks for the α-tocopherol+phospholipid, and the α-tocopherol+phospholipid+polysorbate, than either the Control or the normal α-tocopherol groups. Serum concentrations at baseline and at the end of two weeks are shown in the following table:

| GROUP | BASELINE | 2 WEEKS |
|---|---|---|
| 1 Control | 3.4 μg/mL | 2.3 μg/mL |
| 2 | 3.3 μg/mL | 2.7 μg/mL |
| 3 | 3.5 μg/mL | *4.2 μg/mL |
| 4 | 3.2 μg/mL | *6.4 μg/mL |

The bioavailability improvement relative to the normal a-tocopherol group (Group 2) after 14 days were:
Group 3 55% improvement *
Group 4 137% improvement *

Statistically different at P<0.05

1. A pharmaceutical formulation comprising a pharmaceutical ingredient comprising a combination of polymethoxyflavones and phospholipids, wherein the combination of the polymethoxyflavones and the phospholipids is in the form of a matrix.

2. The pharmaceutical formulation of claim 1, wherein the pharmaceutical ingredient further comprising tocotrienols.

3. The pharmaceutical formulation of claim 1, wherein the polymethoxyflavones is selected from the group consisting of nobiletin, tangeretin, sinensetin, scutellarein and any natural or synthetic derivatives thereof.

4. The pharmaceutical formulation of claim 1, wherein the phospholipids are present in an amount of from about 20% to about 90% by weight of the total dosage form.

5. The pharmaceutical formulation of any of claim 1, wherein the pharmaceutical ingredients are present in an effective amount to treat cardiovascular disease when administered to a human patient.

6. The pharmaceutical formulation of claim 1, wherein the formulation is in the form of tablets, capsules or powder.

7. A pharmaceutical formulation comprising a pharmaceutical ingredient comprising at least about 15% w/w of tangeretin; at least about 15% w/w of nobiletin; at least about 1% of sinensetin; at least about 0.5% w/w of tetramethylscutellarein; at least about 2% w/w of desmthylinobiletin; and at least about 14% w/w tocotrienols.

8. A method of preparing a pharmaceutical ingredient comprising a combination of polymethoxyflavones and phospholipids, wherein the pharmaceutical ingredient is mixed, compressed and extruded under a pressure of at least 30° C. from about 10 seconds to about 90 seconds, with an effective amount of sheer to cosolubilize the polymethoxyflaones into the phospholipids.

9. The pharmaceutical formulation of claim 1, wherein the phospholipids have an acetone insoluble index of about 90% or greater.

10. The method of claim 8, wherein the phospholipids content has an acetone insoluble index of about 90% or greater.

11. The pharmaceutical formulation of claim 2, wherein polymethoxyflavones and the tocotrienols are in a ratio of about 75:25 to about 95:5, the polymethoxyflacones selected from the group consisting of an essence oil isolated from a citrus fruit, a peel oil isolated from a citrus fruit, a peel isolated from a citrus fruit, decharacterized citrus fruit, and combinations thereof.

12. The pharmaceutical formulation of claim 1, wherein the pharmaceutical ingredient further comprises at least one pharmaceutically acceptable excipient.

13. The pharmaceutical formulation of claim 5, wherein the cardiovascular disease is hypercholesterolemia or atherosclerosis.

14. The pharmaceutical formulation of claim 2, wherein the pharmaceutical ingredient combination comprises flavonoids and tocotrienols in a ratio of about 90:10.

15. The pharmaceutical formulation of claim 2, wherein the pharmaceutical ingredient combination comprises flavonoids and tocotrienols in a ratio of about 80:20.

16. The pharmaceutical formulation of claim 2, wherein the active agent combination comprises flavonoids and tocotrienols in a ratio of about 95:5.

17. The pharmaceutical formulation of claim 1, comprising from about 50% to about 90% of the pharmaceutical ingredient combination.

18. The pharmaceutical formulation of claim 1, comprising from about 60% to about 80% of the pharmaceutical ingredient combination.

19. The pharmaceutical formulation of claim 1, comprising from about 70% of the pharmaceutical ingredient combination.

20. The pharmaceutical formulation of claim 6, wherein the formulation is suitable for administration orally.

21-76. (canceled)
